(19) United States
(12) Patent Application Publication
(10) Pub. No.: US 2022/0146607 A1
(43) Pub. Date: May 12, 2022

(54) MAGNETIC RESONANCE IMAGING APPARATUS AND IMAGING MANAGEMENT METHOD

(71) Applicant: Canon Medical Systems Corporation, Otawara-shi (JP)

(72) Inventors: Hitoshi KANAZAWA, Utsunomiya (JP); Seiji NOZAKI, Otawara (JP); Takehiro SHIBUYA, Nasushiobara (JP)

(73) Assignee: Canon Medical Systems Corporation, Otawara-shi (JP)

(21) Appl. No.: 17/519,962

(22) Filed: Nov. 5, 2021

(30) Foreign Application Priority Data
Nov. 9, 2020 (JP) 2020-186611

(51) Int. Cl. G01R 33/38; G01R 33/3815

(52) U.S. Cl. CPC G01R 33/3804; G01R 33/3815

(57) ABSTRACT

According to one embodiment, a magnetic resonance imaging apparatus includes processing circuitry. The processing circuitry calculates a limit imaging condition based on one or more imaging parameters for determining an imaging condition, the limit imaging condition being an allowable limit relating to heat input to a superconducting magnet. The processing circuitry limits an input range of an imaging parameter input by an operator based on the limit imaging condition.

### CROSS-REFERENCE TO RELATED APPLICATIONS

This application is based upon and claims the benefit of priority from Japanese Patent Application No. 2020-186611, filed Nov. 9, 2020, the entire contents of which are incorporated herein by reference.

### FIELD

Embodiments described herein relate generally to a magnetic resonance imaging apparatus and an imaging management method.

### BACKGROUND

Embodiments described herein relate generally to a magnetic resonance imaging apparatus and an imaging management method.

A superconducting magnetic resonance imaging apparatus (superconducting MRI apparatus) uses, for example, helium as a refrigerant for a superconducting coil. However, the recent steep rise in helium prices has put pressure on lifetime costs of an MRI apparatus. Therefore, it is desirable to adopt a low-capacity refrigerant in which a helium capacity is made as small as possible.

The recent attention to low-capacity refrigerants creates the need to consider a phenomenon in which the application of a gradient field in imaging causes an induced current in, for example, a superconducting coil inside a superconducting magnet, thereby resulting in an increased temperature inside the superconducting magnet (Gradient Coil Induced Heating: GCIH). That is, in an MRI apparatus with a large capacity of helium and a sufficient amount of refrigerant there is a high probability that generated heat can be absorbed by evaporation of the refrigerant even with an increase in temperature of a superconducting coil. However, an MRI apparatus with a small amount of refrigerant such as an MRI apparatus adopting a low-capacity refrigerant cannot cope with the rapid increase in GCIH due to imaging, thereby increasing the possibility that quenching will be caused by heat intruding from the outside world.

For this reason, a conventional technique predicts behavior of a magnet regarding quenching for each imaging, and halts the imaging before it is actually performed if the risk of quenching is high. However, a determination as to whether to perform an imaging is made at a stage of executing the imaging after setting an imaging condition. Thus, in the case of a determination that an imaging is not executable, an imaging condition needs to be reset. Accordingly, there is a possibility of causing the need to execute another imaging by changing an imaging condition or being on standby until an imaging is completed, thereby causing a problem wherein an imaging cannot be performed efficiently.

### BRIEF DESCRIPTION OF THE DRAWINGS

FIG. 1 is a conceptual diagram showing an MRI apparatus according to a present embodiment.

FIG. 2 is a flowchart showing imaging management processing of the MRI apparatus according to the present embodiment.

FIG. 3 is a flowchart showing details of processing of estimating a limit imaging condition according to the present embodiment.

FIG. 4 is a conceptual diagram showing an example of calculating the generated heat amount required for generating a generated heat amount database according to the present embodiment.

FIG. 5 is a diagram showing one example of a transfer function according to the present embodiment.

FIG. 6 is a diagram showing an example of a user interface screen according to the present embodiment.

FIG. 7 is a diagram showing a first display example of a user interface relating to an imaging parameter.

FIG. 8 is a diagram showing a second display example of the user interface relating to an imaging parameter.

### DETAILED DESCRIPTION

In general, according to one embodiment, a magnetic resonance imaging apparatus includes processing circuitry. The processing circuitry calculates a limit imaging condition based on one or more imaging parameters for determining an imaging condition, the limit imaging condition being an allowable limit relating to heat input to a superconducting magnet. The processing circuitry limits an input range of an imaging parameter input by an operator based on the limit imaging condition.

A magnetic resonance imaging device (MRI apparatus) and an imaging management method according to a present embodiment will be described with reference to the accompanying drawings. The description of the embodiments will assume that the components or portions having the same reference signs are adapted to operate in the same manner, and redundant explanations will be omitted as appropriate.

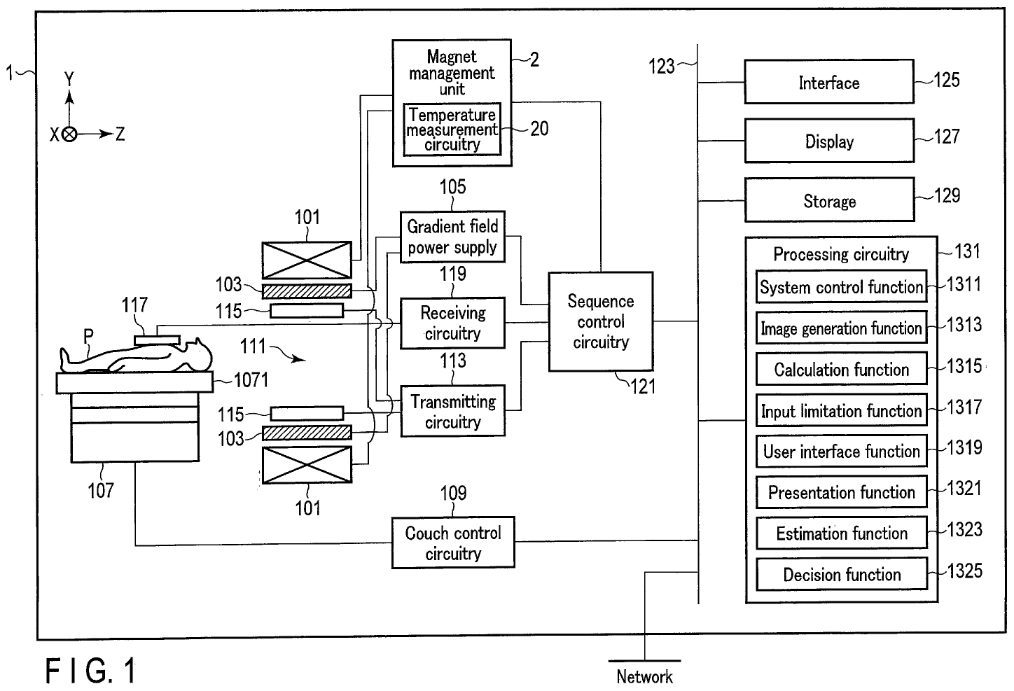

FIG. 1 is a conceptual diagram showing an MRI apparatus according to a present embodiment.

As shown in FIG. 1, an MRI apparatus 1 includes a static magnetic field magnet 101, a magnet management unit 2, a gradient field coil 103, a gradient field power supply 105, a couch 107, couch control circuitry 109, a transmitting circuitry 113, a transmitter coil 115, a receiver coil 117, receiving circuitry 119, sequence control circuitry 121, a bus 123, an interface 125, a display 127, a storage 129, and processing circuitry 131. The MRI apparatus 1 may include a hollow, cylindrical-shaped shim coil provided between the static magnetic field magnet 101 and the gradient field coil 103.

The static magnetic field magnet 101 is a magnet formed in a hollow, approximately cylindrical shape. The static magnetic field magnet 101 is not necessarily in an approximately cylindrical shape; it may be formed in an open shape. The static magnetic field magnet 101 generates a uniform static magnetic field in its inner space. In the present embodiment, the static magnetic field magnet 101 is assumed to be a superconducting magnet using a superconducting coil.

The gradient field coil 103 is a coil formed in a hollow, approximately cylindrical shape. The gradient field coil 103 is arranged inside the static magnetic field magnet 101. The gradient field coil 103 is formed by combining three coils respectively corresponding to the X axis, the Y axis, and the Z axis, which are orthogonal to each other. The Z axis direction is defined as the same as the direction of the static magnetic field. In addition, the Y-axis direction is a vertical direction, and the X-axis direction is a direction perpendicular to each of the Z axis and the Y axis. Each of the three coils of the gradient field coil 103 receives a current from the gradient field power supply 105 and generates a gradient field in which the magnetic field intensity changes along a corresponding one of the X axis, the Y axis, and the Z axis.

The gradient fields along the X axis, the Y axis, and the Z axis generated by the gradient field coil 103 respectively form, for example, a gradient field for frequency encoding (also referred to as a readout gradient field), a gradient field for phase encoding, and a gradient field for slice selection. The gradient field for frequency encoding is used to change a frequency of an MR signal in accordance with a spatial position. The gradient field for phase encoding is used to change a phase of an MR signal in accordance with a spatial position. The gradient field for slice selection is used to determine an imaging cross section.

The gradient field power supply 105 is a power supply device that supplies a current to the gradient field coil 103 under the control of the sequence control circuitry 121.

The couch 107 is a device having a couch top 1071 on which a subject P is placed. The couch 107 inserts the couch top 1071 on which the subject P is placed into a bore 111 under the control of the couch control circuitry 109. The couch 107 is installed in an examination room in which the MRI apparatus 1 is installed, in such a manner that the longitudinal axis of the couch 107 is parallel to the central axis of the static magnetic field magnet 101. The couch control circuitry 109 is circuitry that controls the couch 107, and drives the couch 107 in response to an operator's instructions via the interface 125 to move the couch top 1071 in the longitudinal direction and the vertical direction.

The transmitter coil 115 is an RF coil arranged inside the gradient field coil 103. Upon receipt of a radio frequency (RF) pulse supplied from the transmitting circuitry 113, the transmitter coil 115 generates a transmit RF wave corresponding to a high frequency magnetic field. The transmitter coil 115 is, for example, a whole body coil. The whole body coil may be used as a transmitter and receiver coil. A cylindrical RF shield is arranged between the whole body coil and the gradient field coil 103 to magnetically separate these coils.

The transmitting circuitry 113 supplies an RF pulse corresponding to a Larmor frequency, etc., to the transmitter coil 115 under the control of the sequence control circuitry 121.

The receiver coil 117 is an RF coil provided inside the gradient field coil 103. The receiver coil 117 receives MR signals emitted from the subject P through a high-frequency magnetic field. The receiver coil 117 outputs the received MR signals to the receiving circuitry 119. The receiver coil 117 is a coil array including, for example, one or more, typically, a plurality of coil elements. The receiver coil 117 is, for example, a phased array coil.

The receiving circuitry 119 generates, under the control of the sequence control circuitry 121, a digital MR signal which is digitized complex number data, based on an MR signal output from the receiver coil 117.

Specifically, the receiving circuitry 119 performs various types of signal processing to an MR signal output from the receiver coil 117, and then executes analog-to-digital (A/D) conversion on data to which the various types of signal processing has been performed. The receiving circuitry 119 samples the A/D-converted data. In this manner, the receiving circuitry 119 generates a digital MR signal (hereinafter, referred to as MR data). The receiving circuitry 119 outputs the generated MR data to the sequence control circuitry 121.

The sequence control circuitry 121 controls the gradient field power supply 105, the transmitting circuitry 113, and the receiving circuitry 119, etc. in accordance with an examination protocol output from the processing circuitry 131, and performs an imaging on the subject P. The examination protocol has various types of pulse sequences (also referred to as imaging sequences) in accordance with the examination. The examination protocol defines the magnitude of a current supplied from the gradient field power supply 105 to the gradient field coil 103, a timing of the supply of the current from the gradient field power supply 105 to the gradient field coil 103, the magnitude of an RF pulse supplied from the transmitting circuitry 113 to the transmitter coil 115, a timing when the RF pulse is supplied from the transmitting circuitry 113 to the transmitter coil 115, and a timing when the MR signal is received by the receiver coil 117, etc.

The bus 123 is a transmission path for transmitting data between the interface 125, the display 127, the storage 129, and the processing circuitry 131. The bus 123 may be connected via, for example, a network to various physiological signal measuring devices, an external storage device, and various modalities. For example, the bus is connected to an electrocardiograph (not shown) as a physiological signal measuring device.

The interface 125 includes circuitry that receives various instructions and information inputs by an operator. The interface 125 includes circuitry relating to, for example, a pointing device such as a mouse, or an input device such as a keyboard. The circuitry included in the interface 125 is not limited to circuitry relating to a physical operational component, such as a mouse or a keyboard. For example, the interface 125 may include an electrical signal processing circuitry which receives an electrical signal corresponding to an input operation from an external input device provided separately from the MRI apparatus 1 and outputs the received electrical signal to various circuitry.

The display 127 displays, for example, various types of magnetic resonance (MR) images generated through an image generation function 1313, and various types of information relating to an imaging and image processing, under the control of a system control function 1311 in the processing circuitry 131. The display 127 is, for example, a CRT display, a liquid crystal display, an organic EL display, an LED display, a plasma display, or any other display or a monitor known in this technical field.

The storage 129 stores, for example, MR data filled in a k-space through the image generation function 1313, and image data generated through the image generation function 1313. The storage 129 stores, for example, various types of examination protocols, imaging conditions including a plurality of imaging parameters that define examination protocols, and the like. The storage 129 stores programs corresponding to various functions implemented by the processing circuitry 131. The storage 129 is, for example, a semiconductor memory element such as a random access memory (RAM), a flash memory, etc., a hard disk drive, a solid state drive, or an optical disk. The storage 129 may also be, for example, a driver that reads and writes various types of information on a portable storage medium such as a CD-ROM drive, a DVD drive, a flash memory, etc.

The magnet management unit 2 includes a temperature measurement circuitry 20.

The temperature measurement circuitry 20 measures, using a temperature sensor, the temperature of one or more superconducting coils forming the static magnetic field magnet 101 that generates a static magnetic field.

The processing circuitry 131 includes, as hardware resources, a processor and a memory such as a read-only memory (ROM), a RAM, etc. (not shown), and collectively controls the MRI apparatus 1. The processing circuitry 131 contains the system control function 1311, the image generation function 1313, a calculation function 1315, an input limitation function 1317, a user interface function 1319, a presentation function 1321, an estimation function 1323, and a decision function 1325.

Various functions of the processing circuitry 131 are stored in the storage 129 in a form of a program executable by a computer. The processing circuitry 131 is a processor that reads a program corresponding to each function from the storage 129 and executes the program to realize the function corresponding to the program. In other words, the processing circuitry 131 that has read each program includes, for example, a plurality of functions shown in the processing circuitry 131 in FIG. 1.

FIG. 1 illustrates the case in which various functions are realized in the single processing circuitry 131; however, the processing circuitry 131 may be constituted by a combination of a plurality of independent processors, and the functions may be realized by the processors executing the programs. In other words, each of the above-mentioned functions may be configured as a program, and executed by a single processing circuitry; alternatively, a specific function may be implemented in a dedicated independent program-execution circuitry.

The term “processor” used in the above description refers to, for example, circuitry such as a central processing unit (CPU), a graphics processing unit (GPU), an application specific integrated circuit (ASIC), a programmable logic device such as a simple programmable logic device (SPLD), a complex programmable logic device (CPLD), or a field programmable gate array (FPGA), etc.

The processor reads and executes a program stored in the storage 129 to implement the corresponding function. A program may be incorporated directly in circuitry of the processor, instead of being stored in the storage 129. In this case, the processor reads and executes a program integrated into the circuitry to realize the corresponding function. Similarly, each of the couch control circuitry 109, the transmitting circuitry 113, the receiving circuitry 119, and the sequence control circuitry 121, etc. is also configured as an electronic circuitry such as the above-described processor.

The processing circuitry 131 controls the MRI apparatus 1 through the system control function 1311. Specifically, the processing circuitry 131 reads a system control program stored in the storage 129 and loads it into the memory, thereby controlling each circuitry of the MRI apparatus 1 in accordance with the loaded system control program. For example, the processing circuitry 131 reads an examination protocol from the storage 129 through the system control function 1311, based on the imaging condition input by an operator via the interface 125. The processing circuitry 131 may generate the examination protocol based on the imaging condition. The processing circuitry 131 transmits the examination protocol to the sequence control circuitry 121, thereby controlling imagings with respect to the subject P.

The processing circuitry 131 applies, through the system control function 1311, an excitation pulse in accordance with an excitation pulse sequence, thereby exerting control in such a manner as to apply a gradient field. After execution of the excitation pulse sequence, the processing circuitry 131 acquires, through the system control function 1311, an MR signal from the subject P in accordance with a data acquisition sequence, which is a pulse sequence for acquisition of various types of data, thereby generating MR data.

The processing circuitry 131 fills MR data along a readout direction of the k-space in accordance with the intensity of a readout gradient field through the image generation function 1313. The processing circuitry 131 generates an MR image by executing a Fourier transform on the MR data filled in the k-space. For example, the processing circuitry 131 can generate an absolute value (magnitude) image from complex MR data. Furthermore, the processing circuitry 131 can generate a phase image using real-part data and imaginary-part data in complex MR data. The processing circuitry 131 outputs an MR image such as an absolute value image and a phase image to the display 127 or the storage 129.

The processing circuitry 131 calculates, through the calculation function 1315, an allowable limit value of the heat amount of the superconducting magnet based on one or more imaging parameters for determining an imaging condition.

The processing circuitry 131 limits, through the input limitation function 1317, an input range of imaging parameters input by an operator, based on a limit imaging condition.

The processing circuitry 131 accepts, through the user interface function 1319, input of one or more imaging parameters for determining an imaging condition.

The processing circuitry 131 presents to an operator, through the presentation function 1321, at least one of the limit imaging condition and a conversion value obtained by converting the limit imaging condition into a risk-correlation value.

The processing circuitry 131 estimates, through the estimation function 1323, a feature amount relating to heat input to a static magnetic field magnet in the case of imaging under the imaging condition.

The processing circuitry 131 decides, through the decision function 1325, whether or not the feature amount satisfies the limit imaging condition.

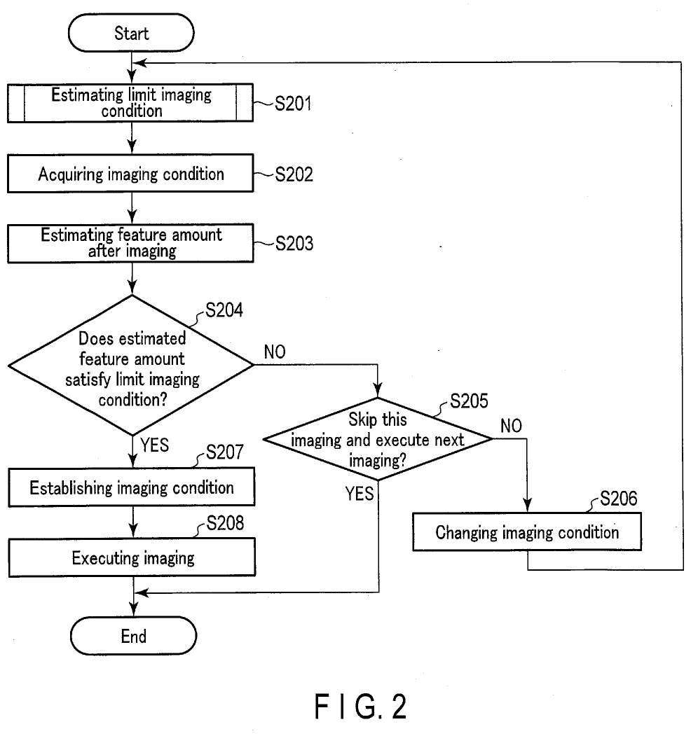

Next, the imaging management processing of the MRI apparatus 1 according to the present embodiment will be described with reference to the flowchart of FIG. 2. The processing shown in the flowchart of FIG. 2 is assumed to be executed when, for example, an imaging condition for one imaging sequence is determined.

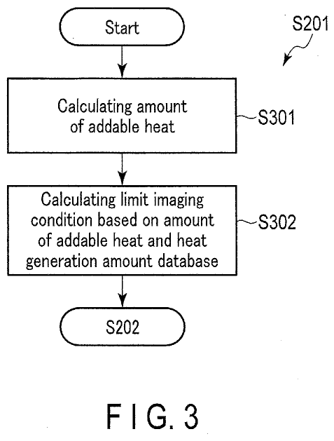

In step S201, the processing circuitry 131 calculates, through the calculation function 1315, a limit imaging condition of settable imaging parameters for an imaging sequence. The method of calculating the limit imaging condition will be described later with reference to FIG. 3 to FIG. 5.

In step S202, the processing circuitry 131 acquires, through the user interface function 1319, an imaging parameter relating to an imaging condition that is operated via a user interface screen or is directly input by an operator. At this time, the processing circuitry 131 limits, through the input limitation function 1317, an input range of imaging parameters input via the user interface screen by the operator, based on the limit imaging condition. Specifically, for example, the processing circuitry sets, through the input limitation function 1317, an upper limit value or a lower limit value of imaging parameters, and sets a limitation in such a manner that values greater than the upper limit value and values smaller than the lower limit value cannot be input.

In step S203, the processing circuitry 131 estimates, through the estimation function 1323, a feature amount relating to a heat amount, which includes the amount of heat generated in the superconducting magnet and is estimated in a case in which an imaging is executed under the imaging condition acquired in step S202. Examples of the feature amount include, in the case of imaging a subject, the amount of heat generated and the amount of temperature change in the superconducting coil, the amount of heat input to the superconducting coil, and so on. Specifically, for example, a value relating to a risk of causing a quenching after imaging a subject may be estimated as a feature amount.

In step S204, the processing circuitry 131 decides, through the decision function 1325, whether or not the feature amount estimated in step S203 satisfies the limit imaging condition. In the case where the feature amount satisfies the limit imaging condition, the processing proceeds to step S207. In the case where the feature amount does not satisfy the limit imaging condition, the processing proceeds to step S205.

In step S205, the processing circuitry 131 decides, through the decision function 1325, whether or not the processing circuitry 131 executes the imaging. For example, in the case where there is an operator's input relating to resetting of an imaging condition, the processing circuitry 131 decides that the condition has been changed, and the processing proceeds to step S206. On the other hand, in the case where an instruction for canceling an imaging is acquired from an operator, the processing circuitry 131 decides that the imaging is not executed (skipped), and terminates the processing.

In step S206, the processing circuitry 131 changes an imaging condition through the user interface function 1319. For example, the processing circuitry 131 receives input relating to the change in an imaging condition from the operator. Furthermore, the processing circuitry 131 may automatically set an alternative imaging condition. Thereafter, the processing returns to step S201, and the same processing is repeated.

An imaging condition acquired in step S202 satisfies the limit imaging condition and the imaging under this imaging condition causes no risk, for example, no quenching. Thus, in step S207, the processing circuitry 131 establishes the imaging condition acquired in step S202.

In step S208, the imaging is executed by the MRI apparatus 1 based on the imaging condition established in step S205.

In step S202, the processing circuitry 131 presents to an operator, through the presentation function 1321, at least one of the limit imaging condition and a conversion value obtained by converting the limit imaging condition into a risk-correlation value. For example, a critical temperature at which the static magnetic field magnet quenches may be displayed as the limit imaging condition, and a temperature gap between the current temperature and the critical temperature of the superconducting magnet or a percentage of the current temperature to the critical temperature may be displayed as the conversion value. This allows an operator to make a reference at the time of inputting the imaging condition.

Alternatively, in step S202, the processing circuitry 131 may preset the imaging condition that satisfies the limit imaging condition. An operator may follow a procedure in which the operator checks preset imaging parameters, and makes an input to make a correction or addition. This can omit the load of inputting all imaging parameters relating to the imaging condition from the beginning.

The processing from step S203 to step S206 may be executed or may be omitted. That is, since the imaging condition acquired in step S202 falls within the range that does not exceed the limit imaging condition, imaging in step S208 may be executed after the processing in step S202.

Next, details of the processing of estimating the limit imaging condition in step S201 will be described with reference to the flowchart of FIG. 3.

In step S301, the processing circuitry 131 calculates, through the calculation function 1315, the amount of addable heat. Specifically, the processing circuitry 131 calculates the amount of heat that can be added (input) without causing a quenching, using e.g., a current temperature of a static magnetic field magnet, a thermal equilibrium temperature of a structure wound with a superconducting wire, a critical temperature at which the static magnetic field magnet quenches, thermal characteristics of the static magnetic field magnet, such as a thermal capacity of a coil portion that generates a main magnetic field by the superconducting wire, and, in the case of a refrigerant such as liquid helium, information on a pressure of the refrigerator.

In step S302, the processing circuitry 131 calculates, through the calculation function 1315, a limit value of imaging conditions, that is, the limit imaging condition based on a heat generation amount database. The heat generation amount database is a database that stores a correspondence relationship between the imaging condition and the amount of generated heat, and is stored in, for example, the storage 129 or an external storage device. Specifically, the processing circuitry 131 calculates, as the limit imaging condition, through the calculation function 1315, a limit value of imaging conditions relating to heat generation such as a repetition time (TR), a slice thickness, and spatial resolution, based on the heat generation amount database, the amount of addable heat calculated in step S301, and the information including the type of imaging and the cross-sectional direction.

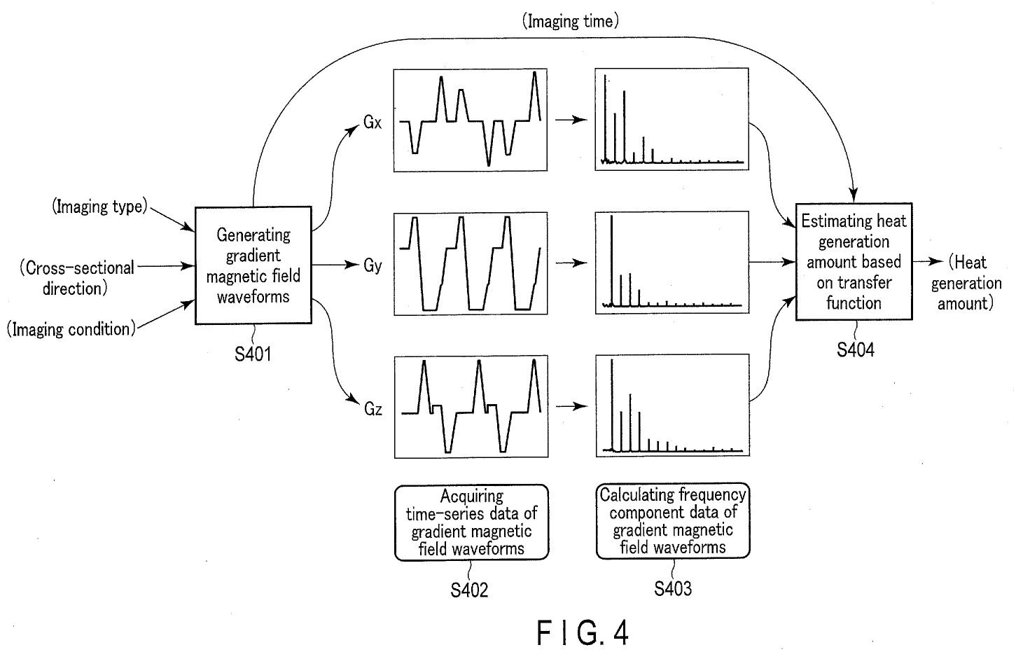

Next, a calculation example of the amount of generated heat required for generating the heat generation amount database will be described with reference to FIG. 4.

For imaging with the MRI apparatus 1, the imaging conditions include, e.g., the type of imaging sequence, the cross-sectional direction, the TR, and the slice thickness described in the above. Various imaging conditions can be considered depending on the imaging purpose. On the other hand, since actual measurement of the amount of generated heat takes a lot of time, it is not realistic to actually measure the amount of generated heat under all the assumed imaging conditions.

Therefore, in step S401 shown in FIG. 4, three gradient magnetic field waveforms in the Gx, Gy, and Gz directions are generated based on the imaging conditions such as the type of imaging method, the cross-sectional direction in imaging, the TR, and the ST.

In step S402, time-series data of the three gradient magnetic field waveforms in the case of imaging according to the imaging sequence is acquired.

In step S403, time-series data of each gradient magnetic field waveform is Fourier transformed, and frequency component data of each gradient magnetic field waveform is calculated.

In step S404, the amount of heat generated by each gradient magnetic field waveform is estimated based on frequency component data of each gradient magnetic field waveform calculated in step S403, by using the transfer function relating to heat generation with respect to each of the gradient magnetic field waveforms of Gx, Gy, and Gz actually measured in advance. By storing the above results in the heat generation amount database, the heat generation amount database can be generated and the amount of generated heat can be calculated according to the imaging conditions.

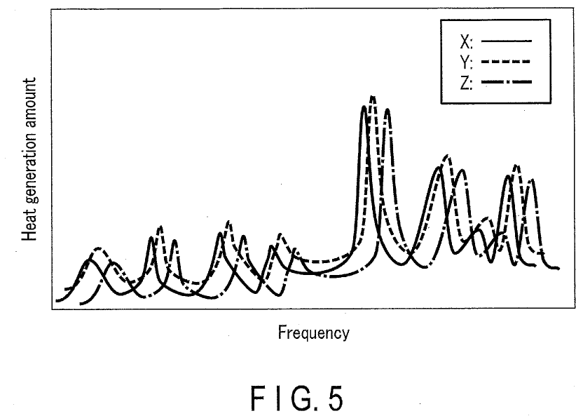

Next, an example of the transfer function used for calculating the amount of generated heat in step S404 will be described with reference to FIG. 5.

FIG. 5 is a graph showing the amount of generated heat for each of the frequency components of the three gradient magnetic fields of Gx, Gy, and Gz. The vertical axis shows the amount of generated heat, and the horizontal axis shows the frequency components.

A relationship of the amount of heat heating a superconducting wire and a support structure of the superconducting wire with respect to the frequency component when, for example, a gradient magnetic field is applied in advance at the time of installing the MRI apparatus 1 or at the time of shipment of the MRI apparatus 1, is measured in the form of the transfer function for each of the three gradient magnetic fields. In order to calculate the transfer function, the amount of generated heat with respect to the imaging condition, which is assumed in an actual imaging, may be calculated in advance.

If the types of imaging sequences are the same, typical imaging conditions such as the TR, the number of slices, and the spatial resolution have a simple correlation with the amount of heat generated. Therefore, by determining a reference imaging condition for each imaging sequence and actually measuring the amount of heat generated at the time of imaging under the determined imaging condition, the limit imaging condition under which imagings can be performed in a state with no risk of quenching can also be determined by making a comparison with the reference imaging condition.

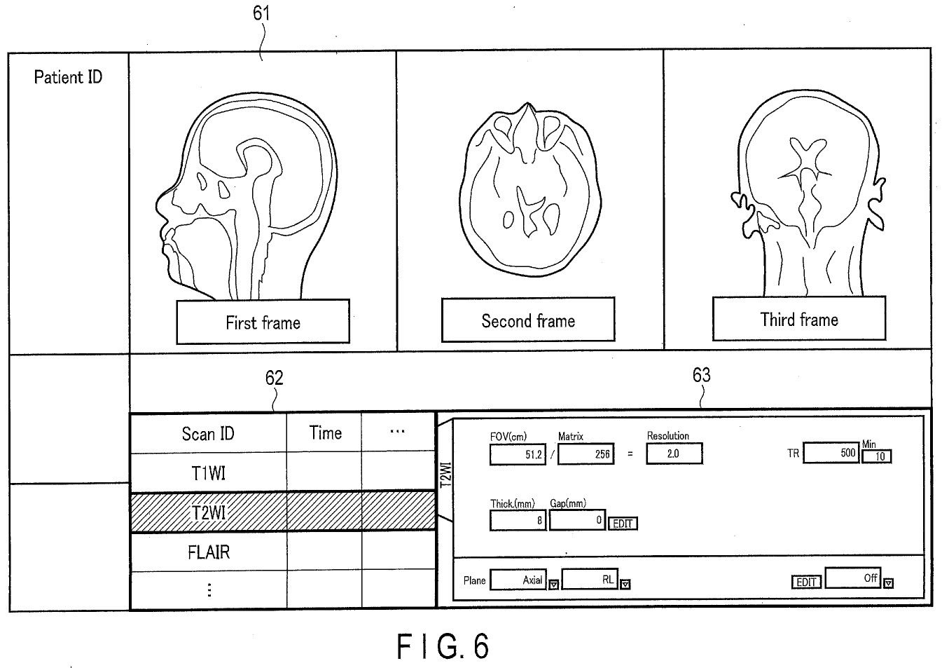

Next, an example of a user interface screen which is also used by the user interface function 1319 will be described with reference to FIG. 6 to FIG. 8.

FIG. 6 shows a user interface screen including an MR image 61 imaged in line with an imaging protocol, a table 62 indicating the type of imaging sequence and an imaging order, and a setting window 63 for setting imaging parameters.

Furthermore, the screen shown in FIG. 6 is in the state in which imaging of the first imaging sequence “T1WI” in the table 62 is completed and an imaging parameter for the second imaging sequence “T2WI” is to be set.

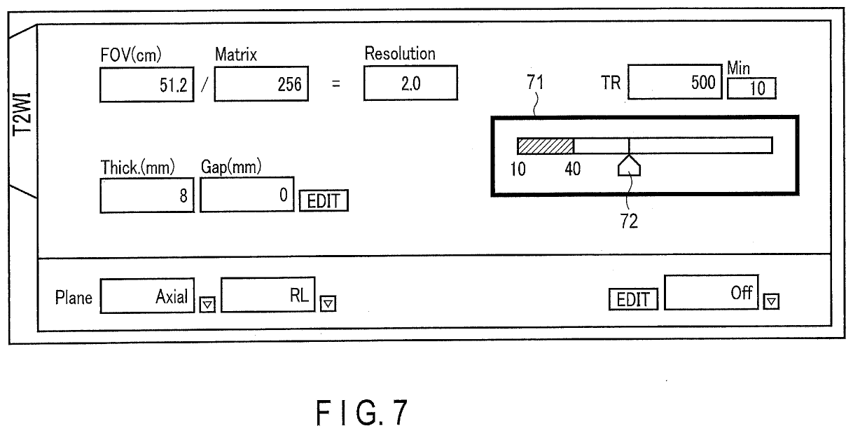

Subsequently, the first display example of the user interface relating to an imaging parameter is shown in FIG. 7. FIG. 7 is an enlarged view of the setting window 63. Herein, it is assumed that an operator inputs a value for the imaging parameter “TR”. For example, when a frame of TR is clicked, a slide bar 71 and a cursor 72 indicating a current value are displayed in another window as the user interface.

In the case where another imaging condition such as the number of slices is fixed, a period in which no gradient magnetic field is output is extended as TR becomes longer, and therefore, the amount of heat generated [W] per unit time decreases. In the direction in which TR becomes shorter, the lower limit is set in such a manner as to prevent the cursor 72 from being slid to a value that does not satisfy the limit imaging condition of a heat input surface, and a value of this lower limit is displayed so that an operator can recognize the lower limit. In the example shown in FIG. 7, unless the limit imaging condition for the heat input surface is considered, the cursor 72 can be moved to “10 ms”; however, in the display, the area located below the lower limit of “40 ms”, which is the limit imaging condition for the heat input surface, is grayed out so that the cursor 72 cannot be moved to this grayed-out area (diagonally shaded part in FIG. 7).

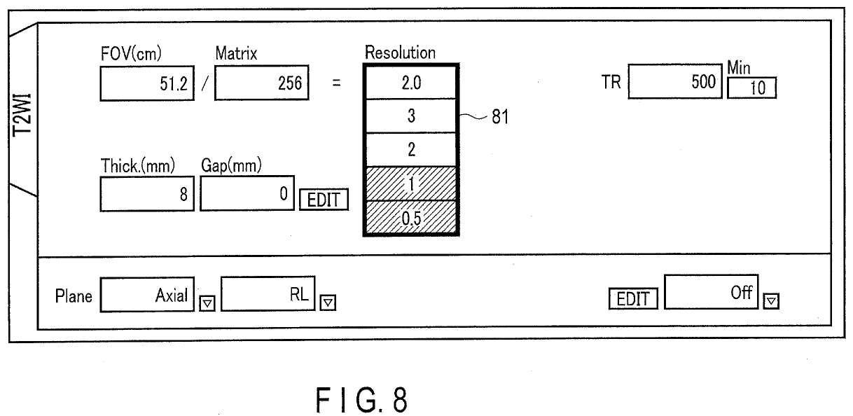

Next, the second display example of the user interface relating to an imaging parameter is shown in FIG. 8. FIG. 8 is also an enlarged view of the setting window 63. Herein, it is assumed that an operator inputs a value for the imaging parameter of “resolution”. For example, when the frame of “resolution” is clicked, a list window 81 is displayed as the user interface.

As a value of the resolution becomes smaller, that is, as the resolution becomes higher, the amount of heat generated in the superconducting magnet becomes larger. Thus, a lower limit is set in such a manner that the direction in which the amount of input heat increases, that is, a small value of resolution which does not satisfy the limit imaging condition, cannot be selected (input), and the lower limit is displayed so that the operator can recognize it. In the example of FIG. 8, assume that the limit imaging condition is set to “2 mm”. Unless this limit imaging condition is considered, the range up to “1 mm” is selectable; however, in the display, the values “1 mm” and “0.5 mm” below the lower limit of “2 mm”, which is the limit imaging condition, are grayed out (diagonally shaded in FIG. 8) so that these values cannot be selected.

In the case of receiving a change of an imaging parameter from an operator through the user interface screen, the processing circuitry 131 may update, through the input limitation function 1317, an input range of another parameter relating to an imaging condition. In the examples shown in FIG. 7 and FIG. 8, the resolution is changed from “3 mm” to “10 mm” based on the limit imaging condition, and it is considered that the limit on the amount of heat input to the superconducting magnet is slightly relaxed. Thus, the imaging parameter “TR” may be updated so that it is changeable to “8000 ms”.

According to the present embodiment described in the above, the limit imaging condition is calculated based on one or more imaging parameters based on the heat generation amount database before the imaging condition is established, and an input range of imaging parameters input by an operator is limited based on the limit imaging condition. In this manner, imaging conditions to be input are limited to a range which allows imagings. This enables imagings to be executed under an input imaging condition. This eliminates the occurrence of resetting of an imaging condition or redoing of imaging at a stage of executing the imaging. As a result, efficient examinations can be supported.

In addition, each function according to the embodiment can also be realized by installing a program for executing the process on a computer such as a workstation and loading it into a memory. The program that causes the computer to execute the processing can be stored and distributed by means of a storage medium, such as a magnetic disk (a hard disk, etc.), an optical disk (CD-ROM, DVD, Blu-ray (registered trademark) etc.), and a semiconductor memory.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. A magnetic resonance imaging apparatus comprising processing circuitry configured to:
calculate a limit imaging condition based on one or more imaging parameters for determining an imaging condition, the limit imaging condition being an allowable limit relating to heat input to a superconducting magnet; and
limit an input range of an imaging parameter input by an operator based on the limit imaging condition.

2. The magnetic resonance imaging apparatus according to claim 1, wherein the processing circuitry is further configured to present to the operator at least one of the limit imaging condition and a conversion value obtained by converting the limit imaging condition into a risk-correlation value.

3. The magnetic resonance imaging apparatus according to claim 1, wherein the processing circuitry is configured to:
set an upper limit value or a lower limit value of the one or more imaging parameters; and
set a limitation in such a manner that values greater than the upper limit value and values smaller than the lower limit value cannot be input.

4. The magnetic resonance imaging apparatus according to claim 1, wherein the processing circuitry is further configured to receive input of one or more imaging parameters for determining an imaging condition from an operator.

5. The magnetic resonance imaging apparatus according to claim 4, wherein when the processing circuitry receives a change of an imaging parameter from the operator, the processing circuitry changes an input range limited in terms of another imaging parameter relating to the imaging condition.

6. The magnetic resonance imaging apparatus according to claim 4, wherein the processing circuitry is further configured to:
display a slide bar through which a value of a target imaging parameter is adjustable; and
set a display in such a manner as to prevent sliding to a value which does not satisfy the limit imaging condition in a direction in which an amount of heat input to the superconducting magnet increases.

7. An imaging management method comprising:
calculating a limit imaging condition based on one or more imaging parameters for determining an imaging condition, the limit imaging condition being an allowable limit relating to heat input to a superconducting magnet; and
limiting an input range of an imaging parameter input by an operator based on the limit imaging condition.

8. The magnetic resonance imaging method according to claim 7, further comprising presenting to the operator at least one of the limit imaging condition and a conversion value obtained by converting the limit imaging condition into a risk-correlation value.

9. The magnetic resonance imaging method according to claim 7, further comprising:
setting an upper limit value or a lower limit value of the one or more imaging parameters; and
setting a limitation in such a manner that values greater than the upper limit value and values smaller than the lower limit value cannot be input.

10. The magnetic resonance imaging method according to claim 7, further comprising receiving input of one or more imaging parameters for determining an imaging condition from an operator.

11. The magnetic resonance imaging method according to claim 10, further comprising changing an input range limited in terms of another imaging parameter relating to the imaging condition when receiving a change of an imaging parameter from the operator.

12. The magnetic resonance imaging method according to claim 10, further comprising:
displaying a slide bar through which a value of a target imaging parameter is adjustable; and
setting a display in such a manner as to prevent sliding to a value which does not satisfy the limit imaging condition in a direction in which an amount of heat input to the superconducting magnet increases.
